(19) United States
(12) Patent Application Publication
(10) Pub. No.: US 2022/0054724 A1
(43) Pub. Date: Feb. 24, 2022

(54) SYSTEM FOR CONTINUOUS RENAL REPLACEMENT THERAPY

(71) Applicant: THE UAB RESEARCH FOUNDATION, Birmingham, AL (US)

(72) Inventors: David ASKENAZI, Birmingham, AL (US); Darryl INGRAM, Birmingham, AL (US); Catherine S. GUROSCKY, Birmingham, AL (US); Kara SHORT, Birmingham, AL (US)

(21) Appl. No.: 17/415,582

(22) PCT Filed: Dec. 23, 2019
(86) PCT No.: PCT/US2019/068389

§ 371 Date: Jun. 17, 2021

Related U.S. Application Data
(60) Provisional application No. 62/783,350, filed on Dec. 21, 2018.

(51) Int. Cl. A61M 1/34; A61M 1/36

(52) U.S. Cl. CPC A61M 1/3431; A61M 1/3609; A61M 1/3606; A61M 2230/20; A61M 1/3672; A61M 2240/00; A61M 2205/36; A61M 1/369

(57) ABSTRACT

Methods and systems for providing continuous renal replacement therapy (CRRT) to a patient are provided. The system includes a hemofiltration unit that provides continuous venovenous hemofiltration (CVVH), a replacement fluid flow regulator configured to regulate the flow of a replacement fluid, an anticoagulant flow regulator configured to pump an anticoagulant, and a blood warmer. The system provides enhanced access to the extracorporeal circuit for blood analysion, infusion of products and/or anticoagulation control. The system may be used to treat patients having renal insufficiency.

### CROSS-REFERENCE TO RELATED APPLICATIONS

This application claims the benefit of U.S. Provisional Application No. 62/783,350, filed Dec. 21, 2018 under the laws of the United States of America and other countries.

### BACKGROUND

### Field of the Disclosure

The present disclosure relates generally to continuous renal replacement therapy (CRRT). More particularly, this disclosure relates to an improved method and system for providing continuous venovenous hemofiltration (CVVH) to a pediatric patient.

### Background

Continuous renal replacement therapy (CRRT) is commonly used to provide renal support for critically ill patients with acute kidney injury. While neonates, infants, and small children experience higher rates of acute kidney injury than most critically ill populations, they receive renal support infrequently. The technical challenges of traditional machines make CRRT initiation in small children very difficult. For instance, most traditional machines are intended for use on adults and utilize a high extracorporeal volume (ECV). When these larger machines are used, the machines require that small children receive CRRT with proportionally larger ECV, filters, blood flows, clearance rates, and vascular catheters. This puts the infant or child at added risk of harm during the CRRT initiation process.

To mitigate concerns posed by CRRT machines with large ECV in relation to blood volume size, the AQUADEX™, a machine designed to perform ultrafiltration in adults with heart failure, has been adapted to provide pre-filter replacement fluids in order to perform CRRT in neonates and small children. The adapted machine maintains electrolyte and water homeostasis, clears waste products, and reduces the ECV, which improves the ability to initiate CRRT in small children who require renal support.

While this modified machine has greatly improved the ability to initiate therapy in neonates and small children, the modified machine does not operate without risk. For instance, the adapted system relies on a pre-replacement intravenous (IV) fluid flow regulator that does not communicate with the extracorporeal pump. If one stops, the other will continue to function absent human intervention. In addition, the current IV fluid delivery systems are designed to operate under low pressure conditions. If the flow regulator delivers fluid into a high pressure system, the amount of delivered fluid can differ. Thus, there is a need for improved access and monitoring of the extracorporeal circuit.

### SUMMARY

The following presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

In a first aspect, a system is provided for CRRT in a patient, the system comprising: a hemofiltration unit comprising a withdrawn blood line connecting a hemofilter to a first catheter connector, a blood pump positioned to propel blood in the withdrawn blood line from the first catheter connector to the hemofilter, a ultrafiltration line connected to receive a filtrate from the hemofilter, an ultrafiltration pump configured to propel the filtrate from the ultrafiltration line by exerting negative pressure on the hemofilter, a return line connected to receive a retentate from the hemofilter and terminating in a second catheter connector, a sampling port in the return line, an infusion port in the withdrawn blood line; an anticoagulant flow regulator configured to regulate the flow of anticoagulant from an anticoagulant source into the withdrawn blood line; a replacement fluid flow regulator configured to regulate the flow of a replacement fluid from a replacement fluid source into one or both of the withdrawn blood line and the replacement blood line; and a blood warmer positioned to warm the retentate prior to infusion into the patient.

In a second aspect, a system is provided for CRRT in a patient, the system comprising: a hemofiltration unit comprising a withdrawn blood line connecting a hemofilter to a first catheter connector; a blood pump positioned to propel blood in the withdrawn blood line from the first catheter connector to the hemofilter; a ultrafiltration line connected to receive a filtrate from the hemofilter; an ultrafiltration pump configured to propel the filtrate from the ultrafiltration line by exerting negative pressure on the hemofilter; a return line connected to receive a retentate from the hemofilter and terminating in a second catheter connector; a sampling port in the return line; a first access connector engaged to the first catheter connector and comprising a first infusion fitting and a first blood draw hub; and a second access connector engaged to the second catheter connector and comprising a second infusion fitting and a second blood draw hub; an anticoagulant flow regulator configured to regulate the flow of anticoagulant from an anticoagulant source into the withdrawn blood line; a replacement fluid flow regulator configured to regulate the flow of a replacement fluid from a replacement fluid source into at least one of the withdrawn blood line and the replacement line; and a blood warmer.

In a third aspect, a system is provided for CRRT in a patient, the system comprising: a hemofiltration unit comprising a withdrawn blood line connecting a hemofilter to a first catheter connector, a blood pump positioned to propel blood in the withdrawn blood line from the first catheter connector to the hemofilter, an ultrafiltration line connected to receive a filtrate from the hemofilter, an ultrafiltration pump configured to propel the filtrate from the ultrafiltration line by exerting negative pressure on the hemofilter, a return line connected to receive a retentate from the hemofilter and terminating in a second catheter connector; and a sampling port in the return line; an anticoagulant flow regulator configured to regulate the flow of anticoagulant from an anticoagulant source into the withdrawn blood line; a replacement fluid flow regulator configured to regulate the flow of a replacement fluid from a replacement fluid source into at least one of the withdrawn blood line and the replacement blood line; a blood warmer positioned to warm the retentate prior to infusion into the patient; and a blood analyzer connected to receive a blood sample from one of the withdrawn blood line and the return line.

In a fourth aspect, a system is provided for CRRT in a patient, the system comprising: a hemofiltration unit comprising a withdrawn blood line connecting a hemofilter to a first catheter connector, a blood pump positioned to propel blood in the withdrawn blood line from the first catheter connector to the hemofilter, an ultrafiltration line connected to receive a filtrate from the hemofilter, an ultrafiltration pump configured to propel the filtrate from the ultrafiltration line by exerting negative pressure on the hemofilter, a return line connected to receive a retentate from the hemofilter and terminating in a second catheter connector, and a sampling port in the return line; an anticoagulant flow regulator configured to regulate the flow of anticoagulant from an anticoagulant source into the withdrawn blood line, said anticoagulant flow regulator comprising an anticoagulant flow regulator control unit configured to control the rate of flow of anticoagulant from the anticoagulant source into the withdrawn blood line; a replacement fluid flow regulator configured to regulate the flow of a replacement fluid from a replacement fluid source into at least one of the withdrawn blood line and the replacement blood line; a blood warmer positioned to warm the retentate prior to infusion into the patient; and a coagulation analyzer connected to receive a blood sample from at least one of the withdrawn blood line and the return line and configured to transmit measurements of coagulation to the anticoagulant flow regulator control unit.

In a fifth aspect, a method for treating renal insufficiency in a patient in need thereof is provided, the method comprising providing continuous renal replacement therapy to the pediatric patient using the system according to the first aspect or second aspect described above.

### BRIEF DESCRIPTION OF THE DRAWINGS

Further features and advantages can be ascertained from the following detailed description that is provided in connection with the drawings described below:

FIG. 1 illustrates an embodiment of the CRRT system comprising a sampling port in the withdrawn blood line and an infusion fitting in the return line.

FIG. 2 illustrates an embodiment of the CRRT system comprising two access connectors connecting the system to the withdrawal and infusion catheters, each of which comprise an infusion fitting and a blood draw port.

FIG. 3 illustrates an embodiment of the CRRT system comprising a blood analyzer (in this case a coagulation analyzer).

FIG. 4 illustrates an embodiment of the CRRT system comprising a control unit for the anticoagulant flow regulator (e.g., a heparin syringe pump) and a coagulation analyzer that feeds data to the anticoagulant's control unit in order to modulate the rate of administration of the anticoagulant.

FIG. 5 is an example of a computing device that may serve as a control unit in the system.

### DETAILED DESCRIPTION

Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. Numerical quantities given herein are approximate unless stated otherwise, meaning that the term “about” or “approximately” can be inferred when not expressly stated.

The terminology used herein is for the purpose of describing particular embodiments only and is not intended to be limiting. As used herein, the singular forms “a,” “an,” and “the” are intended to include the plural forms as well (i.e., at least one of whatever the article modifies), unless the context clearly indicates otherwise.

With reference to the use of the word(s) “comprise” or “comprises” or “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and/or the following claims.

The term “consisting essentially of” means that, in addition to the recited elements, what is claimed may also contain other elements (steps, structures, ingredients, components, etc.) that do not adversely affect the operability of what is claimed for its intended purpose. Such addition of other elements that do not adversely affect the operability of what is claimed for its intended purpose would not constitute a material change in the basic and novel characteristics of what is claimed.

Terms such as “at least one of A and B” should be understood to mean “only A, only B, or both A and B.” The same construction should be applied to a longer list (e.g., “at least one of A, B, and C”).

The terms “first”, “second”, “third,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The terms “treatment”, “treat” and “treating” as used herein refers a course of action initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method or device of the present disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

In this disclosure terms such as “administering”, or “administration” include acts such as prescribing, dispensing, giving, or taking a substance such that what is prescribed, dispensed, given, or taken is actually contacts the patient's body externally or internally (or both). It is specifically contemplated that instructions or a prescription by a medical professional to a subject or patient to take or otherwise self-administer a substance is an act of administration.

System for CRRT

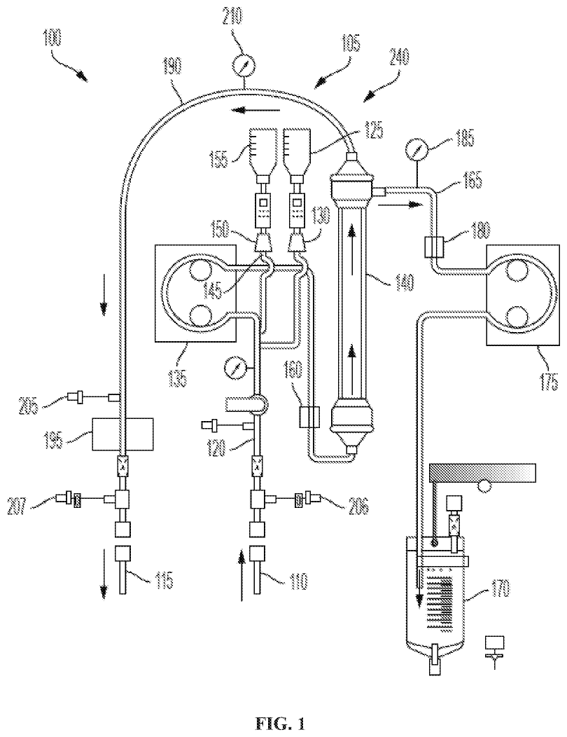

FIG. 1 illustrates the operation and fluid path of the CRRT system 100 according to an exemplary embodiment of the present disclosure. The CRRT system 100 of the present disclosure advantageously provides an extracorporeal circuit 105 having a low extracorporeal volume (ECV) that is particularly suitable for use with infants and small children (although suitable in some instances for larger children and adults). In one embodiment, the ECV of the present extracorporeal circuit 105 is about 20 mL to about 40 mL. In another embodiment, the ECV of the present extracorporeal circuit 105 is about 30 mL to about 40 mL. In still another embodiment, the ECV of the present extracorporeal circuit 105 is about 33 mL. Higher ECV may be used in some instances, for example for non-pediatric applications. To initiate treatment, access points for the withdrawal and return of blood are inserted to suitable peripheral or central veins on a patient. Blood is withdrawn from the patient through a withdrawal access point 110 and returned to the patient through a return access point 115. The withdrawal access point 110 and the return access point 115 may include a needle, a central line, or a catheter. Once the blood is withdrawn from the patient, the blood enters a withdrawn blood line 120 and into a hemofiltration unit 240. The withdrawn blood line 120 may be made of any type of suitable plastic tubing, such as polyvinyl chloride (PVC) tubing.

In some embodiments, the withdrawn blood line 120 is operatively connected to an anticoagulant source 125. “Anticoagulant” as used herein refers to any substance having the effect of inhibiting the coagulation of blood. Examples of anticoagulants include, but are not limited to, heparin, warfarin, rivaroxaban, dabigatran, apixaban, citrate, and edoxaban. The anticoagulant source 125 may be operatively connected to the withdrawn blood line 120 by any suitable connector to provide the anticoagulant. For instance, the anticoagulant source 125 may be operatively connected to the withdrawn blood line 120 by a Y-connector. In a preferred embodiment the anticoagulant source 125 infuses anticoagulant into the withdrawn blood line 120 upstream of the blood pump 136. In one embodiment, an anticoagulant flow regulator 130 is configured to pump the anticoagulant from the anticoagulant source 125 into the withdrawn blood line 120. Such flow regulators are generally referred to in the art as “pumps,” even when they merely control the rate of flow caused by gravity instead of actively pumping the anticoagulant. Suitable “pumps” are commercially available, such as the ALARIS family of pumps (Becton, Dickinson and Company Franklin Lakes, N.J.).

In some embodiments of the system, the withdrawn blood line 120 may be operatively connected to a hematocrit sensor 215. The hematocrit sensor 215 can be configured to measure the volume percentage of red blood cells in the blood in the withdrawn blood line 120. The hematocrit sensor 215 may also be controlled by software that can automatically stop the removal of fluid from the patient if the hematocrit hits a prescribed hematocrit limit.

Blood flow is controlled by a blood pump 135 that is positioned to propel blood in the withdrawn blood line 120 to a filter 140. In one embodiment, the blood pump 135 is a roller pump. In this aspect, as the blood pump 135 rotates, rollers compress a segment of the withdrawn blood line 120 to generate blood flow. The blood pump 135 provides negative pressure through the withdrawn blood line 120 to bring blood from the patient to the filter 140. The blood pump 135 may be rotated by a motor under microprocessor control. In one embodiment, the blood pump 135 may be designed to operate from about 10 mL/minute to about 40 mL/minute. In another embodiment, the blood pump 135 may be designed to operate from about 15 mL/minute to about 35 mL/minute. In still another embodiment, the blood pump 135 may be designed to operate from about 20 mL/minute to about 30 mL/minute.

While blood is pumped through the withdrawn blood line 120, replacement fluid is infused into the withdrawn blood line 120 through a port 145, for instance, a pigtail. In one embodiment, a replacement fluid flow regulator 150 is configured to pump the replacement fluid into the withdrawn blood line 120. Again, such flow regulators are generally referred to in the art as “pumps,” even when they merely control the rate of flow caused by gravity instead of actively pumping the replacement fluid. For example, the replacement fluid flow regulator 150 may be a large volume infusion pump such as the ALARIS™ pump module. In another embodiment, the replacement fluid flow regulator 150 is configured to regulate the flow of the replacement fluid into the withdrawn blood line 120. The replacement fluid may be stored in a replacement fluid source 155 that is operatively connected to the port 145. The replacement fluid can be infused into the withdrawn blood line 120 before or after the blood pump 135. In some embodiments of the system the replacement fluid is infused into the return line 190 post-filter 140 (in theory replacement fluid could be infused at multiple points in the extracorporeal circuit 105).

In some embodiments, as the blood travels from the blood pump 135 to the filter 140, the blood passes through an air detector 160. The air detector 160 can detect air in amounts exceeding about 50 microliters. If an air bubble is detected, the blood pump 135 is stopped instantaneously. In this embodiment, since air can only enter the extracorporeal circuit 105 from the pre-pump segment, the air detector 160 should be positioned before the filter 140. In another embodiment, the air detector 160 should be positioned before or after the filter 140.

As the blood travels from the blood pump 135, the blood passes through a filter 140. The infusion of replacement fluids allows for the use of continuous venovenous hemofiltration (CVVH). In this aspect, the filter 140 is a hemofilter 140 having a semi-permeable membrane. In CVVH, a high rate of ultrafiltration across the semi-permeable hemofilter membrane is created by a hydrostatic gradient, and solute transport occurs by convection (in some cases by negative pressure exerted by an ultrafiltration pump 175). Solutes (small molecular weight solutes) are entrained in the bulk flow of water across the membrane (which is permeable to water), while the membrane prevents red blood cells, proteins, and other blood components from passing through. Unlike methods of hemodialysis, the transfer of solutes is not driven by a concentration gradient between the blood and a low-solute dialysis fluid.

In some embodiments, the hemofilter has an effective filtration area of about 0.09 m² to about 0.3 m². In another embodiment, the hemofilter has an effective filtration area of about 0.1 m² to about 0.2 m². In still another embodiment, the hemofilter has an effective filtration area of about 0.12 m². Higher effective filtration areas may be used in some instances, for example for non-pediatric applications. The hemofilter may be composed of a porous material. For example, the hemofilter may be composed of polysulfone membranes. The hemofilter 140 has a sieving coefficient that allows for the passage of water and small solutes, such as urea, creatinine, and vitamin B12, but prevents the passage of larger components, such as red blood cells, proteins (such as albumin), and other blood components. As used herein, “sieving coefficient” refers to the ratio of solute filtrate concentration to the respective solute plasma concentration. A sieving coefficient of 1 indicates unrestricted transport while a sieving coefficient of zero indicates there is no transport. In one embodiment, the hemofilter 140 has a sieving coefficient for urea of about 0.98. In another embodiment, the hemofilter 140 has a sieving coefficient for creatinine of about 0.98. In still another embodiment, the hemofilter 140 has a sieving coefficient for vitamin B12 of about 0.98. In yet another embodiment, the hemofilter 140 has a sieving coefficient for albumin of less than about 0.02.

Upon completion of the hemofiltration, a filtrate (e.g., the filtered plasma and small solutes) exits the hemofilter 140 and is pumped through an ultrafiltration line 165 to a filtrate collection container 170, such as a collection bag. An ultrafiltration pump 175 may be used to propel the filtrate through the ultrafiltration line 165 by exerting negative pressure on the hemofilter 140. In one embodiment, the ultrafiltration pump 175 can perform ultrafiltration from about 10 mL/h to about 500 mL/h. In another embodiment, the ultrafiltration pump 175 can perform ultrafiltration from about 50 mL/h to about 450 mL/h. In still another embodiment, the ultrafiltration pump 175 can perform ultrafiltration from about 100 mL/h to about 400 mL/h. In some embodiments, ultrafiltration rates of up to 500 mL/h are ample for clearance of waste products in small children in CVVH mode. Higher pump rates may be used in some instances, for example for non-pediatric applications.

A blood leak detector 180 may be operatively connected to the filtrate line 165 and positioned adjacent to the ultrafiltration pump 175. The blood leak detector 180 is used to detect the leakage of red blood cells across the filter 140 membrane if the membrane is damaged. In one embodiment, the blood leak detector 180 may be of a photometric type and respond to the change of color of the filtrate. An ultrafiltrate pressure sensor 185 may also be operatively connected to the filtrate line 165. In one embodiment, the ultrafiltrate pressure sensor 185 is configured to measure pressure in the filtrate line 165. The ultrafiltrate pressure sensor 185 can also be used to monitor the transmembrane pressure (TMP) and to detect clotting or fouling of the filter 140. A withdrawal pressure sensor may also be present to monitor pressure in the withdrawn blood line 120.

Blood exits the hemofilter 140 through a return line 190 and is continuously returned to the patient through the return line 190 that is terminated at the return access point 115. In one embodiment, a blood warmer 195 may be operatively connected to the return line 190. The blood warmer 195 can heat the blood prior to transfusion back into the patient. Thermal management of the extracorporeal circuit 105 is of increased importance in pediatric patients and neonates.

In another embodiment, a sampling port 205 may also be operatively connected to the return line 190. The sampling port 205 can be used to monitor the anticoagulation of the blood that is returned to the patient. In some embodiments, an infusion pressure sensor 210 is operatively connected to the return line 190. The infusion pressure sensor 210 may be configured to measure the pressure in the return line 190.

In the embodiment of the system 100 shown in FIG. 1, an additional sampling/infusion port 206 is present in the withdrawn blood line 120, and an additional sampling/infusion port 207 is present in the return blood line 190. The sampling/infusion ports 206 and 207 may be any that is suitable for withdrawing blood or infusion fluids, preferably aseptically and while avoiding chemical contamination. Such suitable forms of sampling/infusion ports 206 include a watertight fitting, a Luer fitting, a septum that can be pierced by a hypodermic needle, and a blood draw hub. The additional sampling ports 206 and 207 can be used to conveniently take blood samples for analysis and infuse medications, blood products or other fluids.

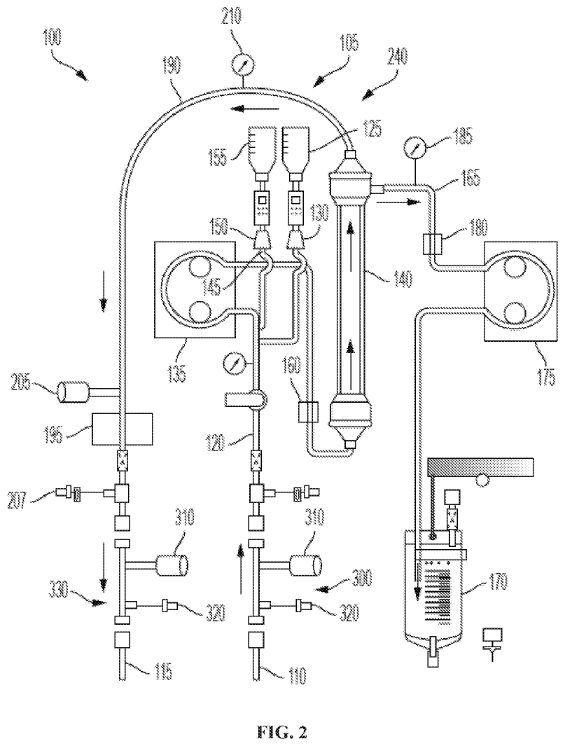

FIG. 2 illustrates an embodiment of the system 100 comprising a first and second access connector 300 & 330. Each access connector 300 & 330 forms a seal with one of the catheter connectors and with a catheter. Blood and retentate pass through an access connector 300 & 330 immediately after withdrawal from the patient and immediate before reinfusion to the patient. The access connector 300 & 330 is a tube suitable to convey blood into and out of the extracorporeal circuit 105 and comprising a blood draw hub 310 & 340 and an infusion fitting 320 & 350. The infusion fitting 320 & 350 may be any that is suitable for permitting the infusion of medications, blood products, fluids or other agents into the extracorporeal circuit 105 and/or the patient. The access connector 300 & 330 has the advantage of allowing the rapid modification of a standard AQUADEX machine to increase the number of infusion fittings 320 & 350 and blood draw hubs 310 & 340.

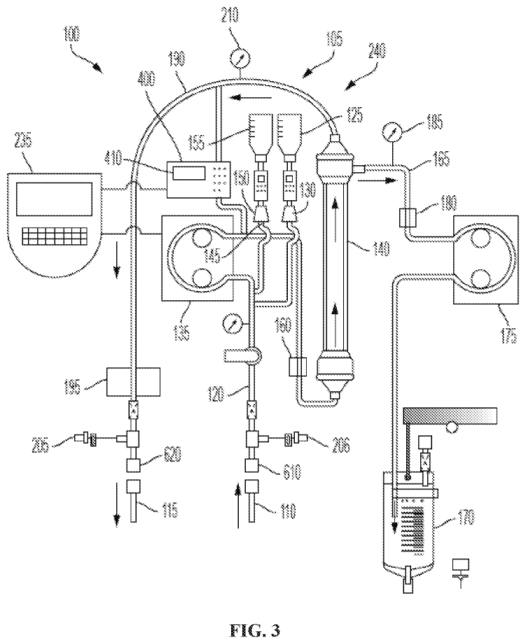

FIG. 3 illustrates an embodiment of the device comprising a blood analyzer 400 connected to receive blood samples from one or both of the withdrawn blood line 120 and the return line 190. The presence of an automated blood analyzer 400 can provide local real-time monitoring of critical aspects of blood chemistry at the bedside. This has the advantage of allowing healthcare personnel to rapidly respond to changes in blood chemistry, rather than waiting for periodic blood samples to be transported to a laboratory for analysis and later reporting. Such real-time monitoring is particularly useful in small pediatric patents, whose blood chemistry is especially vulnerable to small changes in CRRT, and whose total blood volume is smaller than larger patients. Useful types of blood analyzer 400 include an electrolyte analyzer (nonlimiting examples of which include those that operate by direct-ion selective electrode channels assay, immunoassay, photometry, potentiometry (ion-selective electrode), immunoturbidimetry, enzymatic, latex agglutination, optical measurement of reflection intensity of reagent color reaction, optical, and reflectance), a blood gas analyzer, a coagulation analyzer 405 (nonlimiting examples of which include that operate by LED optical means, turbidimetric (for clot detection), aggregation tests, immunologic, chromogenic, simultaneous wavelength scanning and PSI checks, clot detection, optical, and turbidimetric), and an antibiotic analyzer. The blood analyzer 400 may transmit data about the analyte or analytes to a display unit 410, which may be advantageously positioned to be viewed by the healthcare professional attending the patient. In some embodiments of the system 100 the blood analyzer 400 is configured to transmit analysis data to a hemofiltration control unit 235 which controls various aspects of the hemofiltration unit 240, such as the operation of the blood pump 135. In some such embodiments the hemofiltration control unit 235 may change the speed of the blood pump 135 in response to changes in the patient's blood chemistry in order to ensure safe and effective CRRT.

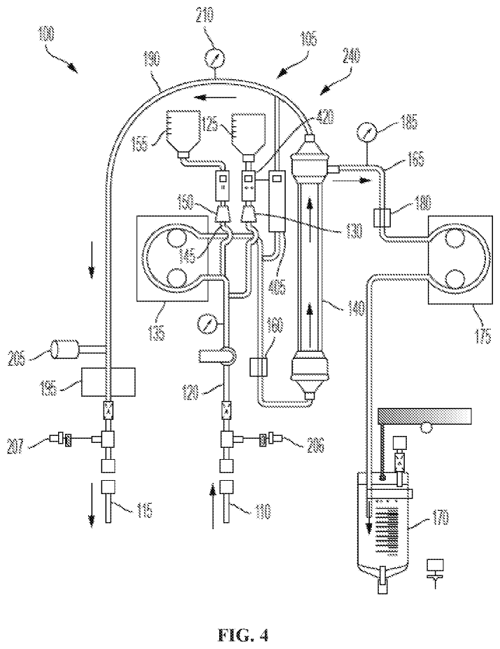

FIG. 4 illustrates an embodiment of the system 100 that includes an anticoagulant flow regulator 130 that controls the rate at which anticoagulant is administered to the withdrawn blood line 120; and a control unit 420 configured to control the anticoagulant flow regulator 130. In some embodiments of the system 100 the anticoagulant flow regulator 130 can be used to modulate the rate of anticoagulant infusion based on the coagulation characteristics of the blood in one or both of the withdrawn blood line 120 and the return line 190. For example, a coagulation analyzer 405 may be present that collects samples from one or both of the withdrawn blood line 120 and the return line 190. The coagulation analyzer 405 may be configured to obtain measurements of coagulation and transmit them to the anticoagulant control unit 420. The anticoagulant control unit 420 may then in turn modulate the rate of infusion of anticoagulant to maintain a target coagulation level, or to simply maintain a steady coagulation level. The measurement of coagulation may include one or more of: partial thromboplastin time (PTT), prothrombin time, thrombin time, complete blood count, anti-factor Xa level, fibrinogen level, and platelet count. In a preferred embodiment the coagulation analyzer 405 measures PTT. In some embodiments of the system the target coagulation level is about 50 to about 70 s PTT. In further embodiments of the system the target coagulation level is about 50 to about 60 s PTT. In further embodiments of the system the target coagulation level is about 0.2 to about 0.4 UI/mL anti-factor Xa.

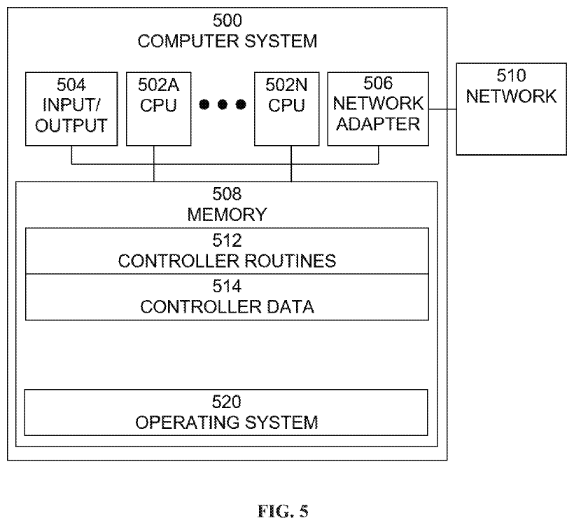

FIG. 5 is a schematic diagram of a computer system 500 of a kind that may be used as a control unit in the systems 100 and methods described herein. Either or both of the hemofiltration control unit 235 and the anticoagulant control 420 unit may be a computer system 500 as shown.

Computer system 500 may typically be implemented using one or more programmed general-purpose computer systems, such as embedded processors, systems on a chip, personal computers, workstations, server systems, and minicomputers or mainframe computers, or in distributed, networked computing environments. Computer system 500 may include one or more processors (CPUs) 502A-502N, input/output circuitry 504, network adapter 506, and memory 508. CPUs 502A-502N execute program instructions to carry out the functions of the present systems 100 and methods. Typically, CPUs 502A-502N are one or more microprocessors, such as an INTEL CORE® processor.

Input/output circuitry 504 provides the capability to input data to, or output data from, computer system 500. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 506 interfaces computer system 500 with a network 510. Network 510 may be any public or proprietary LAN or WAN, including, but not limited to, the Internet.

Memory 508 stores program instructions that are executed by, and data that are used and processed by, CPU 502 to perform the functions of computer system 500. Memory 508 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

Memory 508 may include controller routines 512, controller data 514, and operating system 520. Controller routines 512 may include software routines to perform processing to implement one or more controllers. Controller data 514 may include data needed by controller routines 512 to perform processing. In one embodiment, controller routines 512 may include software for analyzing and communicating incoming data from the hemofiltration control unit 235 (e.g., measurements related to the functioning and speed of the blood pump 135). In another embodiment, controller routines 512 may include software for analyzing and communicating incoming data from the blood analyzer 400 and/or coagulation analyzer 405 (e.g., measurements related to dosing and the rate of infusion).

Methods of Use

The CRRT system 100 described herein can be used, for instance, to treat or support a patient with renal insufficiency by providing one or more kidney functions. The renal insufficiency could have various causes, such as one or more of acute injury, congenital defect, acute kidney disease, intrinsic renal disease, and chronic kidney disease. In some embodiments, the CRRT system 100 described herein may be used to treat acute intrinsic renal disease. In a further embodiment the CRRT system 100 is used to acute toxic renal injury.

In one embodiment, a method for treating or supporting a patient with renal insufficiency in need thereof is provided. In this embodiment, the method includes withdrawing blood from the patient and processing the blood with the CRRT system 100 described herein such that the blood undergoes CVVH. The processed blood (i.e., having passed through the filter 140 of the disclosed CRRT system 100) may then be reinfused into the patient. The disclosed method may be administered to any patient in need of medical care to treat renal insufficiency. In a preferred embodiment the patient is a pediatric patient. A “pediatric patient,” as used herein, refers to a patient less than 18 years of age. In another embodiment, the patient is an infant. An “infant,” as used herein, refers to a patient less than 1 year old. In another embodiment, the patient is a neonate. A “neonate,” as used herein, refers to a patient less than four weeks old.

### Exemplary Embodiments

This description provides support for the following listed embodiments of the subject matter described above, in addition to and without prejudice to what is claimed.

Embodiment 1: A system 100 for providing continuous renal replacement therapy to a patient, the system 100 comprising: a hemofiltration unit 240 comprising a withdrawn blood line 120 connecting a hemofilter 140 to a first catheter connector 610; a blood pump 135 positioned to propel blood in the withdrawn blood line 120 from the first catheter connector 610 to the hemofilter 140; a ultrafiltration line 165 connected to receive a filtrate from the hemofilter 140; an ultrafiltration pump 175 configured to propel the filtrate from the ultrafiltration line 165 by exerting negative pressure on the hemofilter 140; a return line 190 connected to receive a retentate from the hemofilter 140 and terminating in a second catheter connector 620; a sampling port 205 in the return line 190; a sampling port 206 in the withdrawn blood line 120; and an infusion fitting 207 in the return line 190; an anticoagulant flow regulator 130 configured to regulate the flow of anticoagulant from an anticoagulant source 125 into the withdrawn blood line 120; a replacement fluid flow regulator 150 configured to regulate the flow of a replacement fluid from a replacement fluid source 155 into at least one of the withdrawn blood line and replacement line; and a blood warmer 195 positioned to warm the retentate prior to infusion into the patient.

Embodiment 2: The system 100 of embodiment 1, wherein each of the sampling port 205 in the return line 190 and the sampling port 206 in the withdrawn blood line 120 are independently selected from one or more of: a blood draw hub, a septum, and a luer fitting.

Embodiment 3: The system 100 of embodiment 1, wherein each of the infusion fitting 207 in the return line 190 and the infusion fitting in the withdrawn blood line 120 are independently selected from one or more of: a septum, and a luer fitting.

Embodiment 4: A system 100 for providing continuous renal replacement therapy to a patient, the system 100 comprising: a hemofiltration unit 240 comprising a withdrawn blood line 120 connecting a hemofilter 140 to a first catheter connector 610; a blood pump 135 positioned to propel blood in the withdrawn blood line 120 from the first catheter connector 610 to the hemofilter 140; a ultrafiltration line 165 connected to receive a filtrate from the hemofilter 140; an ultrafiltration pump 175 configured to propel the filtrate from the ultrafiltration line 165 by exerting negative pressure on the hemofilter 140; a return line 190 connected to receive a retentate from the hemofilter 140 and terminating in a second catheter connector 620; a sampling port 205 in the return line 190; a first access connector 300 engaged to the first catheter connector 610 and comprising a first infusion fitting 320 and a first blood draw hub 310; and a second access connector 330 engaged to the second catheter connector 620 and comprising a second infusion fitting 350 and a second blood draw hub 340; an anticoagulant flow regulator 130 configured to regulate the flow of anticoagulant from an anticoagulant source 125 into the withdrawn blood line 120; a replacement fluid flow regulator 150 configured to regulate the flow of a replacement fluid from a replacement fluid source 155 into at least one the withdrawn blood line 120 and the replacement line; and a blood warmer 195 positioned to warm the retentate prior to infusion into the patient.

Embodiment 5 : The system 100 of embodiment 4, wherein the first access connector 300 and the second access connector 330 each comprise a fitting to connect to one of a withdrawal access point 110 and a return access point 115.

Embodiment 6: The system 100 of embodiment 4, wherein each of the first and second infusion fittings 350 are independently selected from one or more of: a septum, and a luer fitting.

Embodiment 7: The system 100 of embodiment 4, wherein the first and second access connector 300 & 330 are substantially identical in configuration.

Embodiment 8: A system 100 for providing continuous renal replacement therapy to a patient, the system 100 comprising: a hemofiltration unit 240 comprising a withdrawn blood line 120 connecting a hemofilter 140 to a first catheter connector 610; a blood pump 135 positioned to propel blood in the withdrawn blood line 120 from the first catheter connector 610 to the hemofilter 140; an ultrafiltration line 165 connected to receive a filtrate from the hemofilter 140; an ultrafiltration pump 175 configured to propel the filtrate from the ultrafiltration line 165 by exerting negative pressure on the hemofilter 140; a return line 190 connected to receive a retentate from the hemofilter 140 and terminating in a second catheter connector 620; and a sampling port 205 in the return line 190; an anticoagulant flow regulator 130 configured to regulate the flow of anticoagulant from an anticoagulant source 125 into the withdrawn blood line 120; a replacement fluid flow regulator 150 configured to regulate the flow of a replacement fluid from a replacement fluid source 155 into at least one of the withdrawn blood line 120 and the replacement line; a blood warmer 195 positioned to warm the retentate prior to infusion into the patient; and a blood analyzer 400 connected to receive a blood sample from one of the withdrawn blood line 120 and the return line 190.

Embodiment 9: The system 100 of embodiment 5, wherein the blood analyzer 400 is selected from one or more of: an electrolyte analyzer, an antibiotic analyzer, a blood gas analyzer, and a coagulation analyzer 405.

Embodiment 10: The system 100 of embodiment 5, wherein the blood analyzer 400 is configured to transmit blood chemistry data to a display unit 410.

Embodiment 11: The system 100 of embodiment 5, wherein the blood analyzer 400 is configured to transmit blood chemistry data to a hemofiltration control unit 235 configured to control the blood pump 135.

Embodiment 12: A system 100 for providing continuous renal replacement therapy to a patient, the system 100 comprising: a hemofiltration unit 240 comprising a withdrawn blood line 120 connecting a hemofilter 140 to a first catheter connector 610; a blood pump 135 positioned to propel blood in the withdrawn blood line 120 from the first catheter connector 610 to the hemofilter 140; an ultrafiltration line 165 connected to receive a filtrate from the hemofilter 140; an ultrafiltration pump 175 configured to propel the filtrate from the ultrafiltration line 165 by exerting negative pressure on the hemofilter 140; a return line 190 connected to receive a retentate from the hemofilter 140 and terminating in a second catheter connector 620; and a sampling port 205 in the return line 190; an anticoagulant flow regulator 130 configured to regulate the flow of anticoagulant from an anticoagulant source 125 into the withdrawn blood line 120, said anticoagulant flow regulator 130 comprising an anticoagulant flow regulator control unit 420 configured to control the rate of flow of anticoagulant from the anticoagulant source 125 into the withdrawn blood line 120; a replacement fluid flow regulator 150 configured to regulate the flow of a replacement fluid from a replacement fluid source 155 into at least one of the withdrawn blood line 120 and the return line; a blood warmer 195 positioned to warm the retentate prior to infusion into the patient; and a coagulation analyzer 405 connected to receive a blood sample from at least one of the withdrawn blood line 120 and the return line 190 and configured to transmit measurements of coagulation to the anticoagulant flow regulator control unit 420.

Embodiment 13: The system 100 of embodiment 12, wherein the anticoagulant flow regulator control unit 420 is configured to modulate the rate of flow of anticoagulant in response to the measurements of coagulation.

Embodiment 14: The system 100 of embodiment 12, wherein the anticoagulant flow regulator control unit 420 is configured to modulate the rate of flow of anticoagulant in response to the measurements of coagulation to maintain steady levels of coagulation.

Embodiment 15: The system 100 of embodiment 12, wherein the measurement of coagulation includes at least one of: partial thromboplastin time (PTT), prothrombin time, thrombin time, complete blood count, anti Factor Xa level, fibrinogen level, and platelet count.

Embodiment 16: The system 100 of any one of the embodiments above, wherein the anticoagulant is heparin.

Embodiment 17: The system 100 of any one of the embodiments above, wherein the anticoagulant flow regulator 130 is a syringe pump.

Embodiment 18: The system 100 of any one of the embodiments above, wherein the heparin flow regulator is not integral with the hemofiltration unit 240.

Embodiment 19: The system 100 of any one of the embodiments above, wherein said replacement fluid flow regulator 150 is not integral with the hemofiltration unit 240.

Embodiment 20: The system 100 of any one of the embodiments above, wherein said blood warmer 195 is not integral with the hemofiltration unit 240.

Embodiment 21: The system 100 of any one of the embodiments above, wherein the hemofiltration unit 240 has an extracorporeal circuit 105 volume of about 33 mL.

Embodiment 22: The system 100 of any one of the embodiments above, wherein the hemofilter 140 has an effective filtration area of about 0.09 to about 0.3 m², about 0.12 m², 0.1 to about 0.2 m², or about 0.12 m².

Embodiment 23: The system 100 of any one of the embodiments above, wherein the hemofilter 140 comprises a multiplicity of polysulfone membranes.

Embodiment 24: The system 100 of any one of the embodiments above, wherein the hemofilter 140 has a sieving coefficient for urea of about 0.98.

Embodiment 25: The system 100 of any one of the embodiments above, wherein the hemofilter 140 has a sieving coefficient for creatinine of about 0.98.

Embodiment 26: The system 100 of any one of the embodiments above, wherein the hemofilter 140 has a sieving coefficient for Vitamin B12 of about 0.98.

Embodiment 27: The system 100 of any one of the embodiments above, wherein the hemofilter 140 achieves solute transfer by convection when in use with the system 100.

Embodiment 28: The system 100 of any one of the embodiments above, wherein the hemofiltration unit 240 comprises at least one of: an infusion pressure sensor 210 positioned to measure pressure in the return line 190, an ultrafiltrate pressure sensor 185 positioned to measure pressure in the ultrafiltration line, and a hematocrit sensor 215 positioned to measure hematocrit in the return line 190.

Embodiment 29: The system 100 of any one of the embodiments above, wherein the blood pump 135 is capable of operating over a range of about 10-40, 15-35, 20-30, 20, or 30 mL min⁻¹.

Embodiment 30: The system 100 of any one of the embodiments above, wherein the ultrafiltration pump 175 is capable of operating over a range of about 0-500 mL h 1.

Embodiment 31: The system 100 of any one of the embodiments above, wherein the hemofiltration unit 240 is not configured for countercurrent dialysis.

Embodiment 32: The system 100 of any one of the embodiments above, comprising a Y-connector connected to the heparin source and the withdrawn blood line 120 to provide the heparin to the withdrawn blood line 120.

Embodiment 33: The system 100 of any one of the embodiments above, wherein the anticoagulant source 125 infuses the anticoagulant into the withdrawn blood line 120 upstream of the blood pump.

Embodiment 34: A method for treating a renal insufficiency in a patient in need thereof, comprising:

withdrawing blood from the patient;

processing the blood with the system of any one of the embodiments above; and

reinfusing the processed blood into the patient.

Embodiment 35: The method of embodiment 34, wherein the renal insufficiency is one or more of: acute injury, congenital defect, acute kidney disease, intrinsic renal disease, and chronic kidney disease.

Embodiment 36: The method of any one of embodiments 34-35, wherein the patient is a pediatric patient.

Embodiment 37: The method of any one of embodiments 34-36, wherein the patient is an infant.

Embodiment 38: The method of any one of embodiments 34-37, wherein the patient is a neonate.

### CONCLUSIONS

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

What is claimed is:

1. A system for providing continuous renal replacement therapy to a patient in need thereof, the system comprising:
(a) a hemofiltration unit comprising
(i) a withdrawn blood line 120 connecting a hemofilter 140 to a first catheter connector;
(ii) a blood pump 135 positioned to propel blood in the withdrawn blood line 120 from the first catheter connector to the hemofilter 140;
(iii) a ultrafiltration line connected to receive a filtrate from the hemofilter 140;
(iv) an ultrafiltration pump 175 configured to propel the filtrate from the ultrafiltration line by exerting negative pressure on the hemofilter 140;
(v) a return line connected to receive a retentate from the hemofilter 140 and terminating in a second catheter connector;
(vi) a sampling port in the return line;
(vii) a sampling port in the withdrawn blood line 120; and
(viii) an infusion fitting in the return line;
(b) an anticoagulant flow regulator 130 configured to regulate the flow of anticoagulant from an anticoagulant source 125 into the withdrawn blood line 120;
(c) a replacement fluid flow regulator 150 configured to regulate the flow of a replacement fluid from a replacement fluid source 155 into at least one of the withdrawn blood line and the return line; and
(d) a blood warmer positioned to warm the retentate prior to infusion into the patient.

2. The system of claim 1, wherein each of the sampling port in the return line and the sampling port in the withdrawn blood line 120 are independently selected from one or more of: a blood draw hub, a septum, and a luer fitting.

3. The system of claim 1, wherein each of the infusion fitting in the return line and the infusion fitting in the withdrawn blood line 120 are independently selected from one or more of: a septum, and a luer fitting.

4. A system for providing continuous renal replacement therapy to a patient in need thereof, the system comprising:
(a) a hemofiltration unit comprising
(i) a withdrawn blood line 120 connecting a hemofilter 140 to a first catheter connector;
(ii) a blood pump 135 positioned to propel blood in the withdrawn blood line 120 from the first catheter connector to the hemofilter 140;
(iii) an ultrafiltration line connected to receive a filtrate from the hemofilter 140;
(iv) an ultrafiltration pump 175 configured to propel the filtrate from the ultrafiltration line by exerting negative pressure on the hemofilter 140;
(v) a return line connected to receive a retentate from the hemofilter 140 and terminating in a second catheter connector;
(vi) a sampling port in the return line;
(vii) a first access connector engaged to the first catheter connector and comprising a first infusion fitting and a first blood draw hub; and
(viii) a second access connector engaged to the second catheter connector and comprising a second infusion fitting and a second blood draw hub;
(b) an anticoagulant flow regulator 130 configured to regulate the flow of anticoagulant from an anticoagulant source 125 into the withdrawn blood line 120;
(c) a replacement fluid flow regulator 150 configured to regulate the flow of a replacement fluid from a replacement fluid source 155 into at least one of the withdrawn blood line and the return line; and
(d) a blood warmer positioned to warm the retentate prior to infusion into the patient.

5. The system of claim 4, wherein the first access connector and the second access connector each comprise a fitting to connect to an intravenous catheter.

6. The system of claim 4, wherein each of the first and second infusion fittings are independently selected from one or more of: a septum, and a luer fitting.

7. The system of claim 4, wherein the first and second access connectors are substantially identical in configuration.

8. A system for providing continuous renal replacement therapy to a patient in need thereof, the system comprising:
(a) a hemofiltration unit comprising
(i) a withdrawn blood line 120 connecting a hemofilter 140 to a first catheter connector;
(ii) a blood pump 135 positioned to propel blood in the withdrawn blood line 120 from the first catheter connector to the hemofilter 140;
(iii) an ultrafiltration line connected to receive a filtrate from the hemofilter 140;
(iv) an ultrafiltration pump 175 configured to propel the filtrate from the ultrafiltration line by exerting negative pressure on the hemofilter 140;
(v) a return line connected to receive a retentate from the hemofilter 140 and terminating in a second catheter connector; and
(vi) a sampling port in the return line;
(b) an anticoagulant flow regulator 130 configured to regulate the flow of anticoagulant from an anticoagulant source 125 into the withdrawn blood line 120;
(c) a replacement fluid flow regulator 150 configured to regulate the flow of a replacement fluid from a replacement fluid source 155 into at least one of the withdrawn blood line and the return line;
(d) a blood warmer positioned to warm the retentate prior to infusion into the patient; and
(e) a blood analyzer connected to receive a blood sample from one of the withdrawn blood line 120 and the return line.

9. The system of claim 5, wherein the blood analyzer is selected from one or more of: an electrolyte analyzer, an antibiotic analyzer, a blood gas analyzer, and a coagulation analyzer.

10. The system of claim 5, wherein the blood analyzer is selected from one or more of: an electrolyte analyzer, a blood gas analyzer, and a coagulation analyzer.

11. The system of claim 5, wherein the blood analyzer is configured to transmit blood chemistry data to a display unit.

12. The system of claim 5, wherein the blood analyzer is configured to transmit blood chemistry data to a hemofiltration control unit 235 configured to control the blood pump 135.

13. A system for providing continuous renal replacement therapy to a patient in need thereof, the system comprising:
(a) a hemofiltration unit comprising
(i) a withdrawn blood line 120 connecting a hemofilter 140 to a first catheter connector;
(ii) a blood pump 135 positioned to propel blood in the withdrawn blood line 120 from the first catheter connector to the hemofilter 140;
(iii) an ultrafiltration line connected to receive a filtrate from the hemofilter 140;
(iv) an ultrafiltration pump 175 configured to propel the filtrate from the ultrafiltration line by exerting negative pressure on the hemofilter 140;
(v) a return line connected to receive a retentate from the hemofilter 140 and terminating in a second catheter connector; and
(vi) a sampling port in the return line;
(b) an anticoagulant flow regulator 130 configured to regulate the flow of anticoagulant from an anticoagulant source 125 into the withdrawn blood line 120, said anticoagulant flow regulator 130 comprising an anticoagulant flow regulator control unit configured to control the rate of flow of anticoagulant from the anticoagulant source 125 into the withdrawn blood line 120;
(c) a replacement fluid flow regulator 150 configured to regulate the flow of a replacement fluid from a replacement fluid source 155 into at least one of the withdrawn blood line and the return line;
(d) a blood warmer positioned to warm the retentate prior to infusion into the patient; and
(e) a coagulation analyzer connected to receive a blood sample from at least one of the withdrawn blood line 120 and the return line and configured to transmit measurements of coagulation to the anticoagulant flow regulator control unit.

14. The system of claim 13, wherein the anticoagulant flow regulator control unit is configured to modulate the rate of flow of anticoagulant in response to the measurements of coagulation.

15. The system of claim 13, wherein the anticoagulant flow regulator control unit is configured to modulate the rate of flow of anticoagulant in response to the measurements of coagulation to maintain steady levels of coagulation.

16. The system of claim 13, wherein the measurement of coagulation includes at least one of: partial thromboplastin time (PTT), prothrombin time, thrombin time, complete blood count, anti-factor Xa level, fibrinogen level, and platelet count.

17. The system of any one of claims 1-16, wherein the anticoagulant is heparin.

18. The system of any one of the claims above, wherein the anticoagulant flow regulator 130 is a syringe pump.

19. The system of any one of claims 1-16, wherein the heparin flow regulator is not integral with the hemofiltration unit.

20. The system of any one of claims 1-16, wherein said replacement fluid flow regulator 150 is not integral with the hemofiltration unit.

21. The system of any one of claims 1-16, wherein said blood warmer is not integral with the hemofiltration unit.

22. The system of any one of claims 1-16, wherein the hemofiltration unit has an extracorporeal circuit volume of about 33 mL.

23. The system of any one of claims 1-16, wherein the hemofilter has an effective filtration area of about 0.09 to about 0.3 m².

24. The system of any one of claims 1-16, wherein the hemofilter has an effective filtration area of about 0.1 to about 0.2 m².

25. The system of any one of claims 1-16, wherein the hemofilter 140 has an effective filtration area of about 0.12 m².

26. The system of any one of claims 1-16, wherein the hemofilter 140 comprises a multiplicity of polysulfone membranes.

27. The system of any one of claims 1-16, wherein the hemofilter 140 has a sieving coefficient for urea of about 0.98.

28. The system of any one of claims 1-16, wherein the hemofilter 140 has a sieving coefficient for creatinine of about 0.98.

29. The system of any one of claims 1-16, wherein the hemofilter 140 has a sieving coefficient for Vitamin B12 of about 0.02.

30. The system of any one of claims 1-16, wherein the hemofilter 140 achieves solute transfer by convection when in use with the system.

31. The system any one of claims 1-16, wherein the hemofiltration unit comprises at least one of: an infusion pressure sensor positioned to measure pressure in the return line, an ultrafiltrate pressure sensor 185 positioned to measure pressure in the ultrafiltration line, and a hematocrit sensor positioned to measure hematocrit in the return line.

32. The system of any one of claims 1-16, wherein the blood pump 135 is capable of operating over a range of about 10-40 mL min⁻¹.

33. The system of any one of claims 1-16, wherein the ultrafiltration pump 175 is capable of operating over a range of up to about 500 mL h⁻¹.

34. The system of any one of claims 1-16, wherein the ultrafiltration pump is capable of operating over a range of about 10-500 mL h⁻¹.

35. The system of any one of claims 1-16, wherein the ultrafiltration pump is capable of operating over a range of about 50-450 mL h⁻¹.

36. The system of any one of claims 1-16, wherein the ultrafiltration pump is capable of operating over a range of about 100-400 mL h⁻¹.

37. The system of any one of claims 1-16, wherein the hemofiltration unit is not configured for countercurrent dialysis.

38. The system of any one of claims 1-16, comprising a Y-connector connected to the heparin source and the withdrawn blood line 120 to provide the heparin to the withdrawn blood line 120.

39. The system of any one of claims 1-16, wherein the blood pump is capable of operating at about 10 to about 40 mL/min.

40. The system of any one of claims 1-16, wherein the blood pump is capable of operating at about 15 to about 35 mL/min.

41. The system of any one of claims 1-16, wherein the blood pump is capable of operating at about 20 to about 30 mL/min.

42. The system of any one of claims 1-16, wherein the blood pump is capable of operating at about 20 mL/min.

43. The system of any one of claims 1-16, wherein the blood pump is capable of operating at about 30 mL/min.

44. The system of any one of claims 1-16, wherein the replacement fluid source infuses return fluid into the withdrawn blood line.

45. The system of any one of claims 1-16, wherein the replacement fluid source infuses return fluid into the return line.

46. The system of any one of claims 1-16, wherein the anticoagulant source infuses the anticoagulant into the withdrawn blood line upstream of the blood pump.

47. A method for treating a renal insufficiency in a patient in need thereof, comprising:
withdrawing blood from the patient;
processing the blood with the system of any one of claims 1-16; and
reinfusing the processed blood into the patient.

48. The method of claim 47, wherein the renal insufficiency is one or more of: acute injury, congenital defect, acute kidney disease, intrinsic renal disease, and chronic kidney disease.

49. The method of claim 47, wherein the patient is a pediatric patient.

50. The method of claim 34, wherein the patient is an infant.

51. The method of claim 47, wherein the patient is a neonate.
